(19) United States
(12) Patent Application Publication
(10) Pub. No.: US 2026/0144906 A1
(43) Pub. Date: May 28, 2026

(54) INTELLIGENT DRONE FOR DETECTION, REMOVAL AND MONITORING OF MOLD IN INDOOR SPACES

(71) Applicant: Microchip Technology Incorporated, Chandler, AZ (US)

(72) Inventors: Victor Duicu, Bucuresti (RO); Valentin Stoia, Bucharest (RO)

(73) Assignee: Microchip Technology Incorporated, Chandler, AZ (US)

(21) Appl. No.: 19/025,322

(22) Filed: Jan. 16, 2025

Related U.S. Application Data
(60) Provisional application No. 63/724,992, filed on Nov. 26, 2024.

(51) Int. Cl. A61L 2/24; A61L 2/10; A61L 2/18; G05D 1/648; G05D 101/10; G05D 105/10; G05D 105/80; G05D 107/60; G05D 109/20; G05D 111/30

(52) U.S. Cl. CPC A61L 2/24; A61L 2/10; A61L 2/18; G05D 1/6486; A61L 2103/75; A61L 2202/14; A61L 2202/16; G05D 2101/10; G05D 2105/10; G05D 2105/89; G05D 2107/60; G05D 2109/20; G05D 2111/34

(57) ABSTRACT

There are systems and methods for automatic mold detection, disinfection, and historical documentation. A system comprises: a drone; a data collection module to collect mold diagnostic data from an interior of a structure and configured to be transported by the drone; a disinfectant module to remediate a mold condition and configured to be transported by the drone; a mold diagnostic circuit configured to: diagnose mold conditions based on the mold diagnostic data; instruct the disinfectant module to remediate a portion of the interior structure based on diagnosed mold conditions; and maintain a historic record of mold conditions and remediations.

### RELATED PATENT APPLICATION

This application claims priority to commonly owned United States Provisional Ser. No. 63/724,992 , filed Nov. 26, 2024, the entire contents of which are hereby incorporated by reference for all purposes.

### TECHNICAL FIELD

The present disclosure relates to hazardous condition remediation, in particular, identification and treatment of hazardous conditions associated with building and structures such as molds, bacteria, viruses, without limitation.

### BACKGROUND

Mold can take root in any building or structure and is a high risk health hazard for young children, the elderly, and persons with weakened immune systems. Mold can cause significant structural damage to building structures, including residential and commercial buildings, schools, hospitals, prisons, dormitories, restaurants, retail brick and mortar structures, airports, hotels, without limitation. Ultra violate (UV) light may be used to disinfect molds, bacteria, viruses, without limitation. Mold may be particularly aggressive after a natural disaster or when a building becomes damaged or compromised.

The spread of mold may be prevented periodically searching for and treating small mold colonies. However, visual inspections by the naked eye may fail to identify small mold colonies. Temperature and humidity sensors scan or monitor limited locations.

UV light disinfection systems may be manually applied to treat potential sources of potential mold growth, viruses, bacteria, without limitation. As used herein, the term “mold” is broadly defined to include molds, bacteria, and viruses.

There is a need for systems and methods to efficiently identify and remediate hazardous environmental conditions associated with buildings and structures.

### SUMMARY

Aspects provide an intelligent drone for monitoring of mold growth in indoor spaces. The drone is also used in mapping the building and paired with a computer application to display areas of potential mold growth.

According to an aspect, there is provided a system comprising: a drone; a data collection module to collect mold diagnostic data from an interior of a structure and configured to be transported by the drone; a disinfectant module to remediate a mold condition and configured to be transported by the drone; a mold diagnostic circuit configured to: diagnose mold conditions based on the mold diagnostic data; instruct the disinfectant module to remediate a portion of the interior structure based on diagnosed mold conditions; and maintain a historic record of mold conditions and remediations.

An aspect provides a system as in the preceding paragraph, wherein the data collection module comprises an imaging module configured to collect mold diagnostic data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

An aspect provides a system as in one of the preceding two paragraphs, wherein the data collection module comprises a sample module configured to collect mold diagnostic data comprising: airborne particles, surface borne particles, temperature, humidity, or gas.

An aspect provides a system as in one of the preceding three paragraphs, wherein the disinfectant module comprises a UV light or a disinfectant sprayer.

An aspect provides a system as in one of the preceding four paragraphs, comprising deployment module configured to deploy a module from the drone and retrieve the module to the drone.

An aspect provides a system as in one of the preceding five paragraphs, comprising a drone station, wherein the drone station comprises: a battery charger to receive and charge a drone battery; and a parking space to park a data collection module, or a disinfectant module.

An aspect provides a system as in one of the preceding six paragraphs, wherein the drone station comprises a mold diagnostic laboratory configured to analyze particle samples.

An aspect provides a system as in one of the preceding seven paragraphs, wherein the mold diagnostic circuit comprises an artificial intelligence engine configured to be trained with the mold diagnostic data in at least near real-time to identify a contour of a moldy zone, wherein the mold diagnostic circuit instructs the disinfectant module to remediate within the contour of the moldy zone.

An aspect provides a system as in one of the preceding eight paragraphs, comprising an ultra-wide band tag associated with the drone and an ultra-wide band anchor, whereby the drone is configured to navigate via the ultra-wide band tag and the ultra-wide band anchor.

According to an aspect, there is provided a method comprising: collecting mold diagnostic data from indoor spaces of a building via a data collection module transported by a drone; generating a mold model based on collected mold diagnostic data; diagnosing mold conditions based on the mold model; and instructing a disinfectant module to remediate a portion of the interior structure based on diagnosed mold conditions.

An aspect provides a method as in the preceding paragraph, wherein data collection module is configured to collect mold diagnostic data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

An aspect provides a method as in one of the preceding two paragraphs, wherein data collection module is configured to collect mold diagnostic data comprising: airborne particles or surface borne particles, temperature, humidity, or gas.

An aspect provides a method as in one of the preceding three paragraphs, comprising: deploying a module from the drone; and retrieving the module to the drone.

An aspect provides a method as in one of the preceding four paragraphs, comprising: charging a drone battery via a battery charger of a drone station; and parking a module in a parking space of a drone station.

An aspect provides a method as in one of the preceding five paragraphs, comprising navigating the drone via an ultra-wide band system comprising ultra-wide band anchors and ultra-wide band tags.

An aspect provides a method as in one of the preceding six paragraphs, wherein generating a mold model comprises: creating a building map with coordinates of building structures and features; associating diagnosed mold conditions in the building map; and creating a plan for periodically collecting mold diagnostic data.

An aspect provides a method as in one of the preceding seven paragraphs, wherein generating the mold model comprises: training an artificial intelligence engine with the mold diagnostic data in at least near real-time to identify a contour of a moldy zone; and training an artificial intelligence engine with mold diagnostic data that corrects previous mold diagnostic data, wherein instructing a disinfectant module comprises instructing the disinfectant module to remediate within the contour of the moldy zone.

According to an aspect, there is provided a drone comprising: a data collection module to collect mold diagnostic data from an interior of a structure; a disinfectant module to remediate a mold condition; a mold diagnostic circuit comprising an artificial intelligence engine configured to be trained with the mold diagnostic data in at least near real-time to identify a contour of a moldy zone and configured to: diagnose mold conditions based on the mold diagnostic data; instruct the disinfectant module to remediate a portion of the interior structure within the contour of the moldy zone based on diagnosed mold conditions; and maintain a historic record of mold conditions and remediations.

An aspect provides a drone as in the preceding paragraph, wherein the data collection module comprises an imaging module configured to collect mold diagnostic data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, sound navigation and ranging (SONAR) images, airborne particles, surface borne particles, temperature, humidity, or gas.

An aspect provides a drone as in one of the preceding two paragraphs, wherein the disinfectant module comprises a UV light or a disinfectant sprayer.

### BRIEF DESCRIPTION OF THE DRAWINGS

The figures illustrate examples of systems and methods to efficiently identify and remediate hazardous environmental conditions, including mold, associated with buildings and structures.

FIG. 1 shows a block diagram of a drone for monitoring and treating molds, bacteria, and viruses, without limitation, on indoor or outdoor surfaces of buildings and structures.

FIG. 2 shows a perspective view of a drone equipped with an imaging module.

FIG. 3 shows a perspective view of a drone equipped with a deploying module having a line that is retractable to suspend and deliver tools.

FIG. 4 shows a drone equipped with a UV light suspended by a retractable line.

FIG. 5 shows a drone equipped with a spray module suspended by a retractable line.

FIG. 6 shows a drone equipped with a mold sample module suspended by a retractable line.

FIG. 7 shows a top view of a drone station.

FIG. 8 shows a top view of a building with ultra-wide band anchors and drones navigating the buildings via ultra-wide band tags to disinfect mold within contours of identified moldy surfaces.

FIG. 9 shows a block diagram of a mold diagnostic system.

FIG. 10 shows a flow chart of a method to provide a system for automatic mold detection and disinfection.

FIG. 11 shows a block diagram of a system to provide automatic mold detection and disinfection.

The reference number for any illustrated element that appears in multiple different figures has the same meaning across the multiple figures, and the mention or discussion herein of any illustrated element in the context of any particular figure also applies to each other figure, if any, in which that same illustrated element is shown.

### DESCRIPTION

According to an aspect, there is provided a system for automatic mold detection and disinfection. A computer application may keep track of and display locations and surface areas of potential or actual mold growth. The system may provide an energy efficient method of UV-based disinfection. The system may record past disinfectant actions and locations predisposed to mold growth.

System components may include: a drone for indoor flight, mapping, and navigation; humidity and temperature sensor; gas and mycotoxins sensors; image and face recognition camera; ; servomotor for module orientation; radio frequency (RF) connectivity to the Internet of Things; LiDAR module for navigation and mapping; real time clock calendar; and an audio speaker system.

According to an example, a user turns on a computer application and the drone. The drone navigates the interior of a building using a LiDAR module for navigation and mapping. The drone scans interior surfaces using humidity and temperature sensors to identify areas or zones of potential mold growth, viruses, bacteria, without limitation. Drone cameras take images of the identified surface areas or zones. Where undesirable conditions, such as mold, viruses, bacteria, without limitation, are detected, the drone confirms the indoor space is clear of individuals who could be harmed by UV light. The drone then irradiates and disinfects the identified surface areas and zones with UV light. The identified surfaces areas and zones are documented or registered so that future testing and treatments may be scheduled. After scanning the entire indoor surfaces of the desired structure of building, the drone returns to a starting point and downloads data to computer executing the computer application.

In some applications, the drone may connect to the Internet of Things to gain security and access information so that the drone may open doors to access rooms and spaces within a building. Through the computer application, a user may select particular indoor spaces, rooms, surfaces, without limitation for the drone to scan, monitor, or treat. In addition to or in alterative to UV light, the drone may treat surfaces by spraying a liquid on surfaces, wherein, the liquid may comprise soap, chlorine, iodine, disinfectants, without limitation. The drone may be scheduled to operate within certain hours of the day, for example when the building is closed for business, or when no individuals are expected to be in the building. For example, a school may be scanned during nighttime hours when no students, teachers or staff are in the school buildings. In addition to or in alternative to humidity and temperature sensors, the drone may have gas sensors to detect, mycotoxins, without limitation. If persons are detected by the drone using facial recognition cameras and applications, the drone may use a speaker system to audibly alert those persons and individuals to advise them to vacate the premises so the drone may conduct disinfecting or treating operations. The drone may identify surface areas or zones as candidates for a manual, deep clean, and may skip a treatment of these areas by the drone.

The drone may provide a treatment by applying a lower dose of spray liquid or intensity of UV light for a longer period of time or a higher dose of spray liquid or intensity of UV light for a shorter period of time. Power or energy may be conserved by the drone by treating surface areas or zones identified as having potential mold growth, viruses, bacteria, without limitation, but not the other surfaces. Identified areas may be targeted by the UV light so that sensitive areas or areas subject to being damaged by the UV light may not be damaged. Lower radiation may be compensated by longer exposure time. Power and energy may be efficiently utilized where the area recognized as mold may be targeted by UV light radiation, but areas without mold are not targeted.

The computer application may record a history of treatment actions and areas being monitored. After an area is disinfected or treated it is marked for future verification that the mold colony has been irradicated. Areas of high temperature and humidity but with no visible indications of mold may be identified and marked as a zone of potential mold growth so that more frequent monitoring may be implemented.

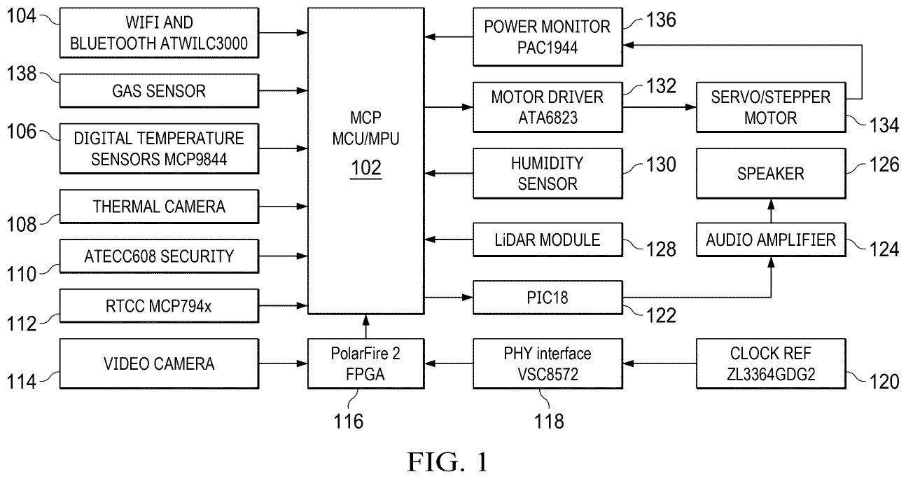

FIG. 1 shows a block diagram of a drone for monitoring and treating molds, bacteria, and viruses, without limitation, on indoor or outdoor surfaces of buildings and structures. The drone has a microcontroller or micro processor unit 102. A Wifi or Bluetooth radio module 104 UWB anchors is connected with the MCP to provide drone communications with other system components, in particular, the computer application and the Internet of Things. Digital temperature sensors 106 are connected with the MCP to record environmental and atmosphere temperature proximate surfaces to provide mold diagnostic data based on detected temperatures. A thermal camera module 108 is connected with the MCP to take images of indoor and outdoor surfaces, objects and individuals, to provide mold diagnostic data based on detected images. A security module 110 is connected to the MCU to provide encrypted and secure communications within and without the drone. A real-time clock and calendar (RTCC) module 112 is connected with the MCP. A video camera module 114 is connected with the MCP 102 through an operating system module 116 to take and record images of surfaces, objects and individuals. A physical layer module 118 and clock reference module 120 are connected with the MCP 102 through the operating system module 116. A controller module 122, an audio amplifier module 124, and a speaker module 126 are connected with the MCP 102 to provide audio warnings and notifications to individuals proximate the drone. A LiDAR module 128 is connected with the MCP 102 to allow the drone to map and navigate indoor spaces. A humidity sensor module 130 is connected with the MCP to allow the drone to detect and record humidity conditions in indoor spaces to collect mold diagnostic data based on detected humidity. A motor driver module 132, a servo/stepper motor 134, and a power monitor module 136 are connected with the MCP 102 to drive and move the drone. A gas sensor 138 is connected to the MCP 102 to provide mold diagnostic data based on detected gasses.

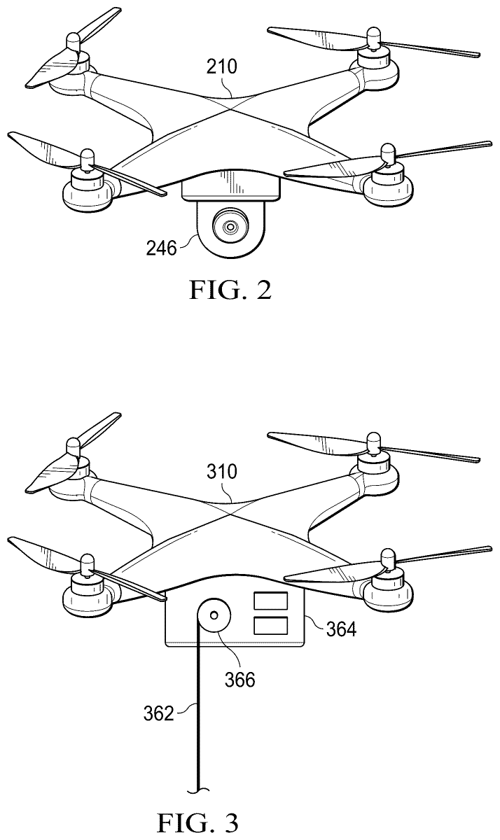

FIG. 2 shows a perspective view of a drone 210 equipped with an imaging module 246, multiple cameras, or a plurality of image sensing devices, and may provide multispectral imaging. The drone 210 may use a navigation system, such as the global position satellite (GPS) system, an ultra-wideband (UWB) navigation system, a Bluetooth Low Energy navigation system, a Wi-Fi navigation system, or a combination of these systems, without limitation. The drone may include global positioning (GPS) with real-time kinematic (RTK) capability, and solar sensors.

Ultra-wideband (UWB) positioning is one example of a technology that may be used to track the position of the drone in a three-dimensional space. UWB positioning transmits short radio pulses across a wide frequency band so that devices may measure the time it takes for a signal to travel between the devices. UWB devices, which utilize a bandwidth of >500 MHz or 20% of the center frequency, may be used to identify a position of a drone using a receiver that is synchronized with a transmitter to determine time separations between pulses in a transmit signal and pulses in a receive signal. Due to its use of relatively short pulses, UWB may enable relatively precise distance and localization detection. UWB positioning calculates the distance between devices with high accuracy and enables location tracking of drones based on the “time of flight” principle using multiple reference points (UWB anchors) to triangulate the position of a drone (tagged with a UWB tag) through either of two processes called Time Difference of Arrival (TDoA) and Two-Way Ranging (TWR).

TDoA utilizes UWB anchors having sensors that are deployed in fixed positions in an indoor space. The sensors of the anchors locate transmitting UWB tracking tags associated with drones within the indoor space. The fixed anchors have synchronized clocks and the UW tags transmit signals in regular intervals. These signals are received and time-stamped by the anchors. All the time-stamped data is then sent to a central processing unit that uses a location engine to analyze the differences in arrival times at the UWB anchors and uses multilateration to calculate the UWB tags'coordinates within the indoor space.

TWR uses two-way communication between two transceivers to sense the distance between them. With TWR, two transceivers range with each other to determine the distance. The time it takes a signal to travel between the transceivers is multiplied by the speed of light and used to determine their relative positions. TWR can be used by fixed UWB anchors and UWB tags, however the TWR process may use one ranging partner to locate the drone at a time.

The imaging module 246 with image sensing devices may provide different spectral sensitivities: ultraviolet (UV), visible spectrum (VIS), near-infrared (NIR), short-wave infrared (SWIR), and thermal, without limitation. Cameras may provide different numbers of bands per camera (mono, color, 2, 4, and 8 band multispectral), and different resolutions per band for example in the megapixel range. The image sensing devices may use light detection and ranging (LIDAR), radio detection and ranging (RADAR), and sound navigation and ranging (SONAR), without limitation.

FIG. 3 shows a perspective view of a drone 310 equipped with a line 362 that is retractable to suspend and deliver tools. The drone 310 is equipped with a tool deployment module 364 comprising a reel 366 and line 362. The tool deployment module 364 works much like a fishing reel and line in that the line 362 may be paid out when the reel 366 is allowed to rotate. The tool deployment module 364 may include a brake to stop, impede, or unimpede rotation of the reel 366 to control whether and how fast the line 362 is to be paid out. A tool (not shown) may be attached to the free end of the line 362, and the weight of the tool and gravity may assist in the line 362 being paid out. If the drone 310 pays out the line 362 until the tool is no longer suspended, but is landed on the ground, the line 362 may be taken in relatively easily by reducing the altitude of the drone 310 as the line 362 is reeled into the tool deployment module 364.

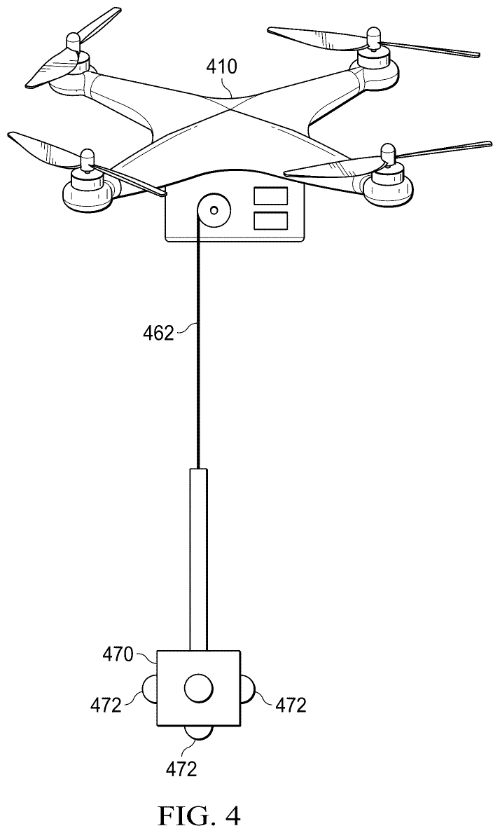

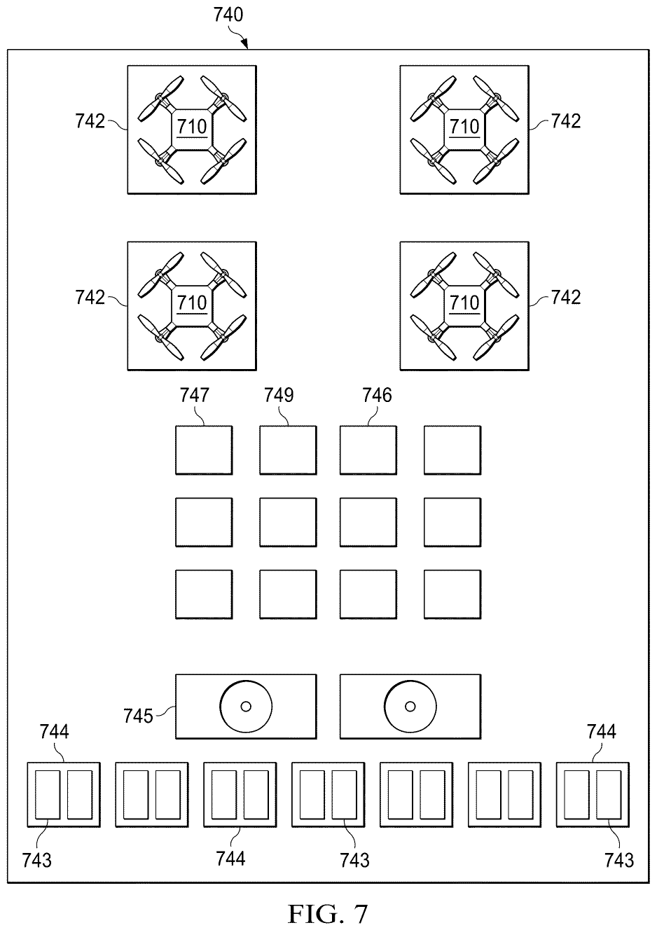

FIG. 4 shows a drone 410 equipped with a UV light 470 suspended by a retractable line 462. The UV light 470 may include one or UV light sources 472 and a power source. When the UV light 470 is allowed to be lowered from the drone 410, the UV light sources 472 may be positioned closer to the identified moldy surface. The UV light 470 may include computer memory to store the mold diagnostic data. Alternatively, the mold diagnostic data may be transmitted to the drone 410 and/or the drone launch pad 740 (see FIG. 7). The mold diagnostic data may be transmitted to the drone 410 via transmission through the line 462 or wirelessly via radio transmission, such as BlueTooth. The mold diagnostic data may be transmitted from the UV light 470 or the drone 410 in real-time, or semi-real-time via wireless radio transmissions. The UV light 470 may be mounted directly to the drone 410 so there is no ability for it to be suspended therefrom. In one aspect, the UV light does not bathe the enire room with UV light, but instead rays of UV light are focused within the contour of a moldy zone

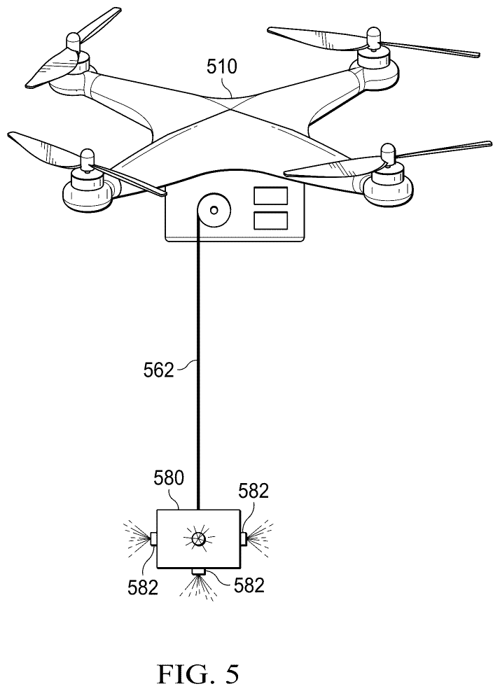

FIG. 5 shows a drone 510 equipped with a liquid disinfectant sprayer 580 suspended by a retractable line 562. The liquid disinfectant sprayer 580 may include one or more nozzles 582 to spray the liquid on moldy surfaces. The liquid disinfectant sprayer 580 may be deployed or lowered from the drone 510, so the liquid disinfectant sprayer 580 may be positioned in closer proximity to the moldy surfaces, and may allow the liquid disinfectant to be sprayed without interference from downdraft of the drone rotors. The liquid disinfectant sprayer 580 may be mounted directly to the drone 510 so there is no ability for it to be suspended therefrom.

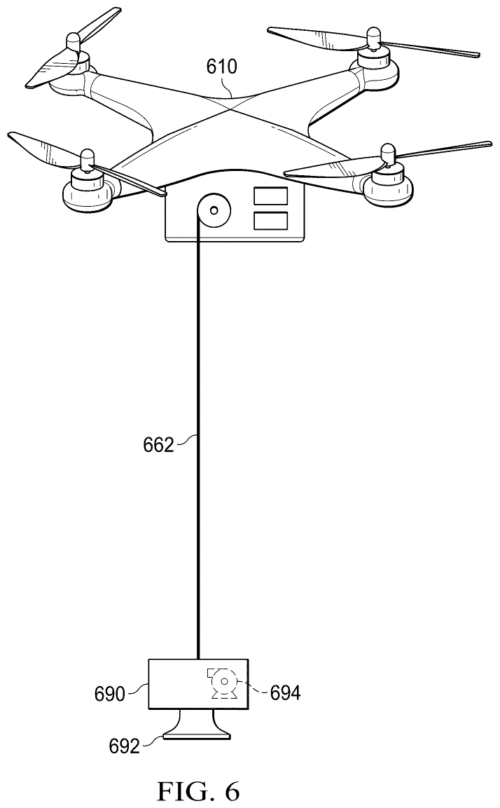

FIG. 6 shows a drone 610 equipped with a mold sample module 690 suspended by a retractable line 662. The mold sample module 690 may include one or more funnels 692 and a pump 694 to vacuum particles from air or surfaces. When the mold sample module 690 is positioned on or proximate a surface with the opening of the funnel 692 exposed to the surface, the pump 694 may suck air and particles into the mold sample module 690. The drone 610 may fly the mold sample to the drone launch pad 740 (see FIG. 7) or another location for analysis. Rather than a vacuum, the mold sample module 690 may also use a cloth or swab to wipe particles from a surface. The mold sample module 690 may be mounted directly to the drone 610 so there is no ability for it to be suspended therefrom.

FIG. 7 shows a top view of a drone launch pad 740. The drone launch pad 740 may be deposited as a stand-alone unit. The drone launch pad 740 may comprise imaging modules 746, disinfectant modules 747, and collection modules 749, without limitation. The imaging modules 746 may comprise the imaging module 246 shown in FIG. 2. The disinfectant modules 747 may comprise the UV light 470 shown in FIG. 4 or the liquid disinfectant sprayer 580 shown in FIG. 5. The collection modules 749 may comprise the mold sample vacuum 690 shown in FIG. 6 or it may comprise a device that collects a sample by wiping or contacting a material with the surface. The drone launch pad 740 may include mold sample receptacles 745, which may automatically test and analyze mold samples upon receipt. The drone launch pad 740 may also include drone batteries 743 and chargers 744. Drones 710 may automatically deposit and retrieve imaging modules 746, disinfectant modules 747, collection modules 749, and batteries 743, without limitation. The mold sample receptacles 745 may include a mold laboratory to automatically analyze samples and provide results to the drone launch pad 740.

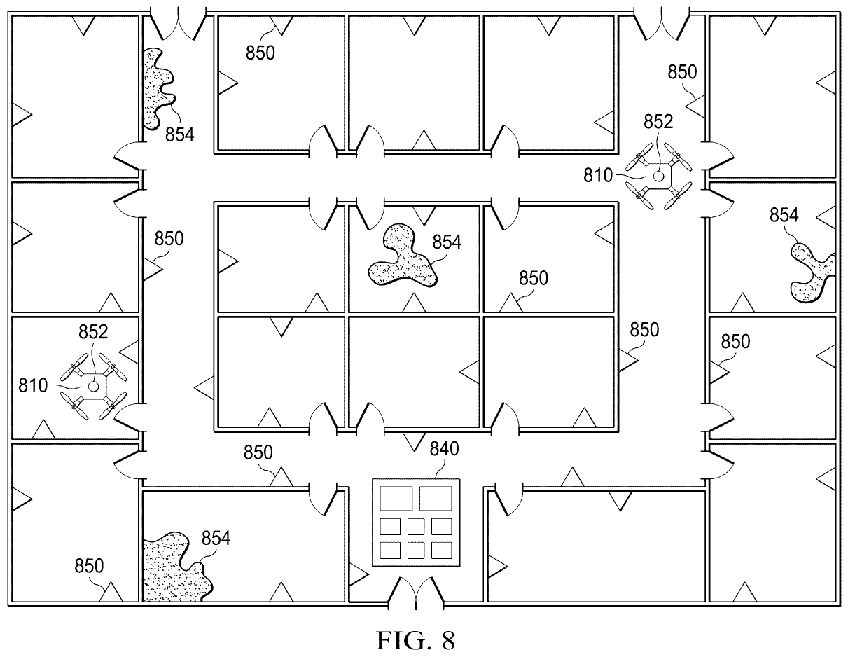

FIG. 8 shows a top view of an indoor building, such as a school, office suite, or hospital. A drone launch pad 840 is positioned in a hallway near the entrance of the building. Drones 810 are in flight within the building, one in a room and the other in a hallway. UWB anchors 850 are positioned on the walls, ceilings, or floors of the interior spaces. The drones 810 may be equipped with UWB tags 852. The UWB anchors 850 transmit short radio pulses across a wide frequency band to the UWB tags 852 so the drones 810 may measure the time it takes for a signal to travel between the devices. The drones 810 calculate the distance between devices with high accuracy and track their locations based on the “time of flight” principle using multiple reference points (UWB anchors 850) to triangulate the drone's position. The drones 810 may use imaging modules, mold sample modules, collection modules, and sensors to collect mold diagnostic data from various locations throughout the building and use the mold diagnostic data to identify a contour of moldy zone 854. The drones 810 may return to the drone launch pad 840 to exchange and/or acquire disinfectant modules, fly to the contour of moldy zone 854 and apply disinfectant remediation (UV light and/or liquid disinfectant) to the surfaces within the contour of moldy zone 854.

The drones may fly in a pattern within an indoor space to collect mold diagnostic data for the entire building, or they may fly to specific locations to collect mold diagnostic data for the specific locations. Mold diagnostic data may include: particle samples, visual images, humidity information, temperature information, without limitation.

Additionally, a robot dog drone could be used instead of a flying drone to significantly reduce power consumption and improve the ability to do continuous operations.

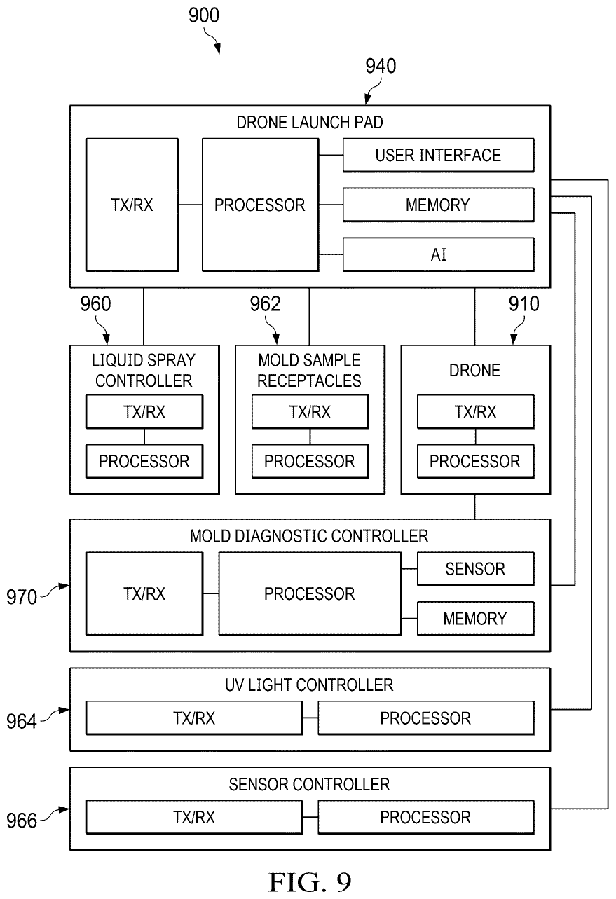

FIG. 9 shows a block diagram of a mold diagnostic and remediation system 900. The system 900 comprises a drone launch pad 940, a liquid spray controller 960, a mold sample receptacle 962, a drone 910, a sensor controller 966, a UV light controller 964, and a mold diagnostic controller 970, wherein the components have a transmitter/receiver to communicate data between the components. The drone launch pad 940 may also have a user interface, memory, and an artificial intelligence engine.

The systems and methods of this disclosure provide “nutritious data” to artificial intelligence engines to inform mold remediation treatments. For example, with reference to FIG. 9, a drone 910 has a data collection module to collect mold data from a building. A sprayer controlled by the liquid spray controller 960 is to spray a condition treating fluid. A drone station 940 is to receive mold diagnostic data from the data collection module of the drone 910 and comprises an artificial intelligence engine of a mold remediation model circuit configured to: diagnose mold conditions based on the mold diagnostic data; instruct the disinfectant module to remediate a portion of the interior structure based on diagnosed mold conditions; and maintain a historic record of mold conditions and remediations. The artificial intelligence engine and the mold remediation model circuit may be implemented by instructions for execution by a processor, analog circuitry, digital circuitry, control logic, digital logic circuits programmed through hardware description language, application specific integrated circuits (ASIC), field programmable gate arrays (FPGA), programmable logic devices (PLD), or any suitable combination thereof, whether in a unitary device or spread over several devices. The artificial intelligence mold remediation model circuit may be implemented by instructions for execution by a processor through, for example, a function, application programming interface (API) call, script, program, compiled code, interpreted code, binary, executable, executable file, firmware, object file, container, assembly code, or object. For example, the artificial intelligence mold remediation model circuit may be implemented by instructions stored in a non-transitory medium such as a memory that, when loaded and executed by a processor such as a CPU (or any other suitable process), cause the functionality of the artificial intelligence mold remediation model circuit described herein.

FIG. 10 shows a flow chart of a method to provide “nutritious data” to artificial intelligence engines to inform the disinfectant module. Mold diagnostic data is collected 1002 from surfaces of a building via a data collection module transported by a drone. The mold diagnostic data may be collected from indoor spaces of a building via a data collection module transported by a drone. An artificial intelligence mold remediation model is generated 1004 based on collected mold diagnostic data. A mold model may be generated based on collected mold diagnostic data. Mold conditions are diagnosed 1006 based on the mold diagnostic data. Mold conditions may be diagnosed based on the mold model. A sprayer is instructed 1008 to spray condition treating fluid on a portion of the building surfaces based on diagnosed mold conditions. A disinfectant module may be instructed to remediate a portion of the interior structure based on diagnosed mold conditions.

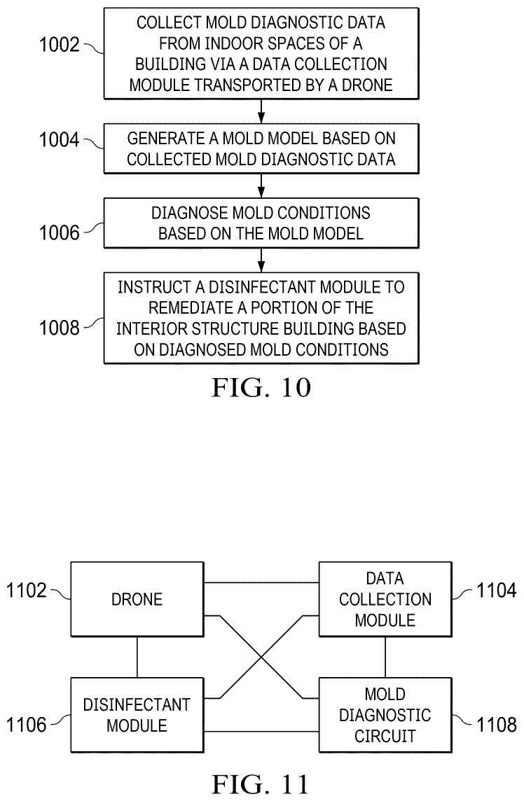

FIG. 11 shows a block diagram of a system for automatic mold detection and disinfection. The system may have a drone 1102. A data collection module 1104 may be provided to collect mold diagnostic data from an interior of a structure and configured to be transported by the drone. A disinfectant module 1106 may be provided to remediate a mold condition and configured to be transported by the drone. A mold diagnostic circuit 1108 may be configured to: diagnose mold conditions based on the mold diagnostic data; instruct the disinfectant module to remediate a portion of the interior structure based on diagnosed mold conditions; and maintain a historic record of mold conditions and remediations.

Although examples have been described above, other variations and examples may be made from this disclosure without departing from the spirit and scope of these disclosed examples.

1. A system comprising:
a drone;
a data collection module to collect mold diagnostic data from an interior of a structure and configured to be transported by the drone;
a disinfectant module to remediate a mold condition and configured to be transported by the drone;
a mold diagnostic circuit configured to:
diagnose mold conditions based on the mold diagnostic data;
instruct the disinfectant module to remediate a portion of the interior structure based on diagnosed mold conditions; and
maintain a historic record of mold conditions and remediations.

2. The system of claim 1, wherein the data collection module comprises an imaging module configured to collect mold diagnostic data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

3. The system of claim 1, wherein the data collection module comprises a sample module configured to collect mold diagnostic data comprising: airborne particles, surface borne particles, temperature, humidity, or gas.

4. The system of claim 1, wherein the disinfectant module comprises a UV light or a disinfectant sprayer.

5. The system of claim 1, comprising deployment module configured to deploy a module from the drone and retrieve the module to the drone.

6. The system of claim 1, comprising a drone station, wherein the drone station comprises:
a battery charger to receive and charge a drone battery; and
a parking space to park a data collection module, or a disinfectant module.

7. The system of claim 6, wherein the drone station comprises a mold diagnostic laboratory configured to analyze particle samples.

8. The system of claim 1, wherein the mold diagnostic circuit comprises an artificial intelligence engine configured to be trained with the mold diagnostic data in at least near real-time to identify a contour of a moldy zone, wherein the mold diagnostic circuit instructs the disinfectant module to remediate within the contour of the moldy zone.

9. The system of claim 1, comprising an ultra-wide band tag associated with the drone and an ultra-wide band anchor, whereby the drone is configured to navigate via the ultra-wide band tag and the ultra-wide band anchor.

10. A method comprising:
collecting mold diagnostic data from indoor spaces of a building via a data collection module transported by a drone;
generating a mold model based on collected mold diagnostic data;
diagnosing mold conditions based on the mold model; and
instructing a disinfectant module to remediate a portion of the indoor spaces based on diagnosed mold conditions.

11. The method of claim 10, wherein data collection module is configured to collect mold diagnostic data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, or sound navigation and ranging (SONAR) images.

12. The method of claim 10, wherein data collection module is configured to collect mold diagnostic data comprising: airborne particles or surface borne particles, temperature, humidity, or gas.

13. The method of claim 10, comprising:
deploying a module from the drone; and
retrieving the module to the drone.

14. The method of claim 10, comprising:
charging a drone battery via a battery charger of a drone station; and
parking a module in a parking space of a drone station.

15. The method of claim 10, comprising navigating the drone via an ultra-wide band system comprising ultra-wide band anchors and ultra-wide band tags.

16. The method of claim 10, wherein generating a mold model comprises:
creating a building map with coordinates of building structures and features;
associating diagnosed mold conditions in the building map; and
creating a plan for periodically collecting mold diagnostic data.

17. The method of claim 10, wherein generating the mold model comprises:
training an artificial intelligence engine with the mold diagnostic data in at least near real-time to identify a contour of a moldy zone; and
training an artificial intelligence engine with mold diagnostic data that corrects previous mold diagnostic data,
wherein instructing a disinfectant module comprises instructing the disinfectant module to remediate within the contour of the moldy zone.

18. A drone comprising:
a data collection module to collect mold diagnostic data from an interior of a structure;
a disinfectant module to remediate a mold condition;
a mold diagnostic circuit comprising an artificial intelligence engine configured to be trained with the mold diagnostic data in at least near real-time to identify a contour of a moldy zone and configured to:
diagnose mold conditions based on the mold diagnostic data;
instruct the disinfectant module to remediate a portion of the interior of the structure within the contour of the moldy zone based on diagnosed mold conditions; and
maintain a historic record of mold conditions and remediations.

19. The drone of claim 18, wherein the data collection module comprises an imaging module configured to collect mold diagnostic data comprising: ultraviolet (UV) images, visible spectrum (VIS) images, near-infrared (NIR) images, short-wave infrared (SWIR) images, thermal images, light detection and ranging (LIDAR) images, radio detection and ranging (RADAR) images, sound navigation and ranging (SONAR) images, airborne particles, surface borne particles, temperature, humidity, or gas.

20. The drone of claim 18, wherein the disinfectant module comprises a UV light or a disinfectant sprayer.
